(19) United States
(12) Patent Application Publication
(10) Pub. No.: US 2025/0101472 A1
(43) Pub. Date: Mar. 27, 2025

(54) GENETICALLY ENGINEERED BACTERIUM FOR THE PRODUCTION OF 3-HYDROXYBUTYRATE

(71) Applicant: Alliance for Sustainable Energy, LLC, Golden, CO (US)

(72) Inventors: Jonathan LO, Westminster, CO (US); Jonathan Richard HUMPHREYS, Denver, CO (US); Wei XIONG, Golden, CO (US); Katherine Jenyan CHOU, Lakewood, CO (US)

(21) Appl. No.: 18/833,797

(22) PCT Filed: Jan. 30, 2023
(86) PCT No.: PCT/US2023/061553

§ 371 Date: Jul. 26, 2024

Related U.S. Application Data
(60) Provisional application No. 63/304,352, filed on Jan. 28, 2022.

(51) Int. Cl. C12P 7/42; C12N 9/04; C12N 9/10

(52) U.S. Cl. CPC C12P 7/42; C12N 9/0006; C12N 9/13; C12Y 101/01157; C12Y 208/03009

(57) ABSTRACT

Disclosed herein are novel methods and compositions of matter to produce 3HB in acetogens by using a(S)-3-hydroxybutyryl-CoA dehydrogenase, Hbd2, responsible for endogenous 3HB production. In conjunction with the heterologous thiolase atoB and CoA transferase ctfAB, hbd2 overexpression improves yields of 3HB on both sugar and syngas (CO/H₂/CO₂), outperforming previously disclosed pathways.

### CROSS-REFERENCE TO RELATED APPLICATIONS

This application is a section 371 national phase entry of PCT application no. PCT/US2023/061,553 filed on 30 Jan. 2023 and also claims priority under 35 U.S.C. § 119 to U.S. provisional patent application No. 63/304,352 filed on 28 Jan. 2022, the contents of which are hereby incorporated in their entirety.

### CONTRACTUAL ORIGIN

The United States Government has rights in this invention under Contract No. DE-AC36-08GO28308 between the United States Department of Energy and Alliance for Sustainable Energy, LLC, the Manager and Operator of the National Renewable Energy Laboratory.

### BACKGROUND

3-Hydroxybutyrate (3HB) is a product of interest as it is a precursor to the commercially produced bioplastic polyhydroxybutyrate. It can also serve as a platform for fine chemicals, medicines, and biofuels, making it a value-added product and feedstock. Acetogens non-photosynthetically fix CO₂ into acetyl-CoA and have been previously engineered to convert acetyl-CoA into 3HB. However, as acetogen metabolism is poorly understood, those engineering efforts have had varying levels of success.

### SUMMARY

In an aspect, disclosed herein is a non-naturally occurring Clostridium sp. comprising a heterologous ctfAB gene and a hbd2 gene. In an embodiment, the hbd2 gene expresses a (S)-3-hydroxybutyryl-CoA dehydrogenase having greater than 70% sequence identity to SEQ ID NO: 2. In an embodiment, the (S)-3-hydroxybutyryl-CoA dehydrogenase uses NADH as a co-factor. In an embodiment, the non-naturally occurring Clostridium sp. further comprises a heterologous atoB gene that is operably linked to the heterologous ctfAB gene. In an embodiment, the heterologous atoB gene that is operably linked to the heterologous ctfAB gene are both integrated into the chromosome of the Clostridium sp. In an embodiment, the specific activity of the (S)-3-hydroxybutyryl-CoA dehydrogenase is up to 0.07 mmol (S)-3-hydroxybutyryl-CoA per minute per milligram. In an embodiment, the Clostridium sp. is Clostridium ljungdahlii.

In an aspect, disclosed herein is a method for making (S)-3-hydroxybutyrate comprising providing a carbon source to a solution comprising a non-naturally occurring Clostridium sp. comprising a heterologous ctfAB gene and a hbd2 gene. In an embodiment, the hbd2 gene expresses a (S)-3-hydroxybutyryl-CoA dehydrogenase having greater than 70% sequence identity to SEQ ID NO: 2. In an embodiment, the (S)-3-hydroxybutyryl-CoA dehydrogenase uses NADH as a co-factor. In an embodiment, the non-naturally occurring Clostridium sp. further comprises a heterologous atoB gene that is operably linked to the heterologous ctfAB gene. In an embodiment, the heterologous atoB gene that is operably linked to the heterologous ctfAB gene are both integrated into the chromosome of the Clostridium sp. In an embodiment, the non-naturally occurring Clostridium sp. makes (S)-3-hydroxybutyrate at a rate up to is 0.083 g/L/hr for 24 hours. In an embodiment, the non-naturally occurring Clostridium sp. makes (S)-3-hydroxybutyrate at up to a concentration of 12 mM. In an embodiment, the non-naturally occurring Clostridium sp. makes (S)-3-hydroxybutyrate at a rate that is up to 2.5-fold greater than a naturally occurring Clostridium sp. In an embodiment, the non-naturally occurring Clostridium sp. is Clostridium ljungdahlii. In an embodiment, the specific activity of the (S)-3-hydroxybutyryl-CoA dehydrogenase is up to 0.07 mmol (S)-3-hydroxybutyryl-CoA per minute per milligram. In an embodiment, the carbon source is syngas. In an embodiment, the carbon source is sugar. In an embodiment, the solution is anaerobic.

Other objects, advantages, and novel features of the present invention will become apparent from the following detailed description of the invention when considered in conjunction with the accompanying drawings.

### BRIEF DESCRIPTION OF THE DRAWINGS

FIGS. 1A, 1B, and 1C depict pathways and enzymes for acetogenic 3HB production. FIG. 1A depicts (S)-3-Hydroxybutyrate production pathway via Hbd1. FIG. 1B depicts (R)-3-Hydroxybutyrate production pathway via CoA transferase/3Hbdh. FIG. 1C depicts the novel (S)-3-Hydroxybutyrate production pathway via CoA transferase Hbd2.

FIG. 2 depicts heterotrophic 3HB production by Clostridium ljungdahlii strains expressing different 3HB pathways. Carbon distribution of fermentation products is indicated by the bar chart %, while the number label is the mM amount after 5 days of growth. In addition to the thiolase (thl) and CoA transferase, four genes were expressed on separate replicating plasmids. A codon optimized 3-Hydroxybutyrate dehydrogenase (3hbdh) from R. sphaeroides (Rsph), from C. difficile (Cdiff), native and codon optimized, and Clju_C23220 were expressed. Note that the C. ljungdahlii gene expressed is not a true 3HB Dehydrogenase. A separate plasmid containing only C. kluyveri hbd1 and C. acetobutyulicum thiolase (thl) was also expressed. Error bars show standard error of the mean (SEM) for 3 biological replicates.

FIGS. 3A, and 3B depict integration of atoB and ctfAB into the pyrE locus of Clostridium ljungdahlii to generate the integrated 3HB strain. FIG. 3A depicts genome integration of atoB and ctfAB into pyrE locus, driven by P_{fdx} in green. P1 & P2 are primer binding sites for screening genomic pyrE locus. FIG. 3B depicts PCR confirmation of integrated atoB and ctfAB using primers P1/P2 to amplify genomic locus. Lanes 1-5 contain screened 5FOA colonies, C1 and C2 are control gDNA from wild type DNA. A successful integration generates a size of 3.8 kb (Lane 2), whereas the wild type size is 1.2 kb (Lane 1, C1, and C2). The numbers next to the DNA ladder indicate kb size.

FIG. 4 depicts a heterotrophic fermentation product profile of Clostridium ljungdahlii strains expressing hbd1 vs. hbd2 in the 3HB strain background. Carbon distribution of fermentation products is indicated by the bar chart percent while the number label is the mM amount after 5 days of growth. The integrated 3HB strain contains the atoB from Escherichia coli and CoA transferase from C. acetobutylicum integrated into the genome. Replicating plasmids expressing a combination of thiolase, hbd1, and hbd2 were transformed into the integrated 3HB strain and measured for 3HB production. The expression of hbd2 results in superior 3HB production. Ckl for C. kluyveri, Clj for C. ljungdahlii. Carbon distribution to 3HB out of the total products is given as a percentage above bars. Error bars show standard error of the mean (SEM) for 3 biological replicates.

FIGS. 5A, 5B, and 5C depict (R) vs. (S) 3HB Pathways and quantification. FIG. 5A depicts the R-3HB pathway FIG. 5B depicts the S-3HB pathway. Red indicates heterologous genes, blue native genes, green native or heterologous. FIG. 5C depicts R (in green) vs. S (in purple) 3HB distribution from heterotrophic fermentation samples using HPLC and enzymatic analysis.

FIGS. 6A, 6B, and 6C depict autotrophic 3HB production in bottle fermentations. Strains were grown on syngas in 250 mL pressure bottles with 70% CO, 10% H₂, and 20% CO₂. FIG. 6A depicts acetate production, FIG. 6B depicts ethanol production, and FIG. 6C depicts 3HB production. Carbon distribution to 3HB out of the total products is given as a percentage above bars. Error bars show standard error of the mean (SEM) for 3 biological replicates.

FIGS. 7A, 7B depict Autotrophic bioreactor fermentation product profile of Clostridium ljungdahlii integrated 3HB strain expressing additional thl and hbd2. The strain contains the atoB thiolase from Escherichia coli and CoA transferase from C. acetobutylicum integrated into the genome with an additional thl2 and hbd2 overexpressed on a replicating plasmid. FIG. 7A depicts optical density over time. FIG. 7B depicts fermentation product profile over time in mM carbon.

### DETAILED DESCRIPTION

Disclosed herein are novel methods and compositions of matter to produce 3HB in acetogens. Disclosed herein is a (S)-3-hydroxybutyryl-CoA dehydrogenase gene hbd2 (SEQ ID NO:1) that expresses a (S)-3-hydroxybutyryl-CoA dehydrogenase (SEQ ID NO: 2) that is responsible for endogenous 3HB production. In conjunction with the heterologous thiolase atoB and CoA transferase ctfAB, hbd2 overexpression improves yields of 3HB on both sugar and syngas (CO/H₂/CO₂), outperforming other tested pathways.

3HB is a chiral bioproduct of interest, with a variety of uses and applications. 3HB can be used for the synthesis of fine chemicals, medicines, biofuels, and bioplastics, especially polyhydroxybutyrate (PHB), which is a highly biodegradable bioplastic. 3HB can also be co-polymerized with other biodegradable polymers to extend their use case and improve physical properties.

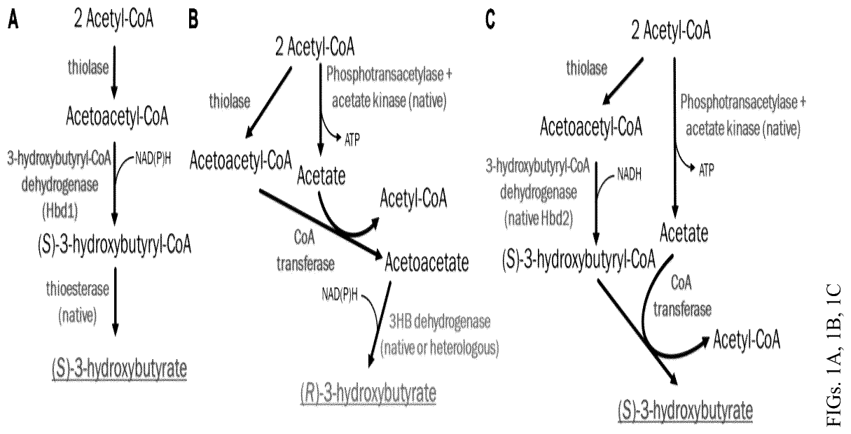

There are two main pathways to generate 3HB in acetogens, with varying levels of success (see FIG. 1). These two pathways will be referred to by their critical genes as hbd1 ((S)-3-hydroxybutyryl-CoA dehydrogenase) (FIG. 1A), and ctfAB (CoA transferase)/3hbdh (3-hydroxybutyrate dehydrogenase) (FIG. 1B). Both pathways require a thiolase to combine two acetyl-CoA molecules into acetoacetyl-CoA, however, from here the pathways diverge. After the formation of acetoacetyl-CoA, Hbd1 reduces acetoacetyl-CoA to (S)-3-hydroxybutyryl-CoA which is subsequently converted to 3HB via a thioesterase through removal of CoA. Conversely, the ctfAB/3hbdh pathway first uses the CtfAB to convert acetoacetyl-CoA to acetoacetate by transferring the CoA to acetate. From here, acetoacetate is then reduced by the 3Hbdh resulting in 3HB (FIG. 1). The hbd1 pathway showed 3HB high titers from syngas, while ctfAB/3hbdh, showed some ability to produce 3HB. The (R)-3-hydroxybutyryl-CoA dehydrogenase gene phaB, from Cupravidus necator, has also been utilized but showed poor 3HB production compared to the other pathways. Disclosed herein is a novel 3HB production pathway in the acetogen Clostridium ljungdahlii.

Disclosed herein is a new pathway for making 3HB in acetogens that relies on ctfAB and an endogenous hbd2, referred to as ctfAB/hbd2 (FIG. 1C). Annotated as (S)-3-hydroxybutyryl-CoA dehydrogenase (EC 1.1.1.57), little was previously known about hbd2 (SEQ ID NO: 1). Hbd2 (SEQ ID NO: 2) is an NADH specific enzyme, however, the functions and applications of this enzyme are not well known. Disclosed herein are methods for using hbd2 for in vivo targeted product formation to improve 3HB titers from syngas and other carbon sources along with additional applications of this enzyme.

### Materials and Methods

### Microbial Strains and Media Composition

Clostridium ljungdahlii DSM 13528 and Clostridium kluyveri DSM 555 were from The Leibniz Institute DSMZ (Germany). Clostridium acetobutylicum ATCC 824 was from American Type Culture Collection (Manassas, VA, United States). C. ljungdahlii growth manipulations were based on previously reported techniques. Routine growth was performed at 37° C. in modified YTF media (10 g/L yeast extract, 16 g/L Bacto tryptone, 4 g/L sodium chloride, 5 g/L fructose, 0.5 g/L cysteine, pH 6). YT media was the same as previous, omitting fructose as a carbon source. Bacterial manipulations were performed in a COY chamber (COY lab, Grass Lake, MI, United States) maintained anaerobic via palladium catalyst with 95% N₂ and 5% H₂ from Airgas (Randor, PA, United States). In general, chemical reagents were purchased from Sigma-Aldrich (St. Louis, MO, United States) or Thermo Fisher (Waltham, MA, United States), unless otherwise indicated.

### Molecular Techniques

Standard molecular cloning and PCR techniques were used with enzymes from New England Biolabs (Ipswich, MA, United States). Routine PCR was performed using Phusion polymerase. For routine cloning and plasmid propagation, Escherichia coli strain NEB 10-Beta was utilized from New England Biolabs. The 1 kb Opti-DNA Marker ladder was from Applied Biological Materials (Vancouver, Canada). Primers and C. ljungdahlii optimized genes were generated from IDT (Coralville, IA, United States). Codon optimized genes atoB from E. coli, 3HBDH from Rhodobacter sphaeroides, the 3HBDH from Clostridium difficile were generated from the IDT algorithm using the Clostridium acetobutylicum option, which has a similar codon usage as C. ljungdahlii. The pMTL80000 plasmids were from Chain Biotech (Nottingham, United Kingdom). Plasmids were generated using Gibson assembly from NEB. Confirmation of plasmids was performed by whole plasmid sequencing from the MGH DNA Core Facility (Cambridge, MA, United States).

Preparation of electrocompetent cells and transformation was performed based on previously reported protocols. Briefly, cells were grown overnight on YTF containing 40 mM DL-Threonine to an OD of 0.2-0.7, harvested, then washed with ice cold SMP buffer (270 mM sucrose, 1 mM MgCl₂, 7 mM sodium phosphate, pH 6), then resuspended in SMP buffer with 10% (dimethyl sulfoxide) DMSO and frozen at −80° C. until transformed. Cells mixed with 2-10 μg of DNA in a 1 mm cuvette, then transformed using a Bio-Rad Gene Pulser Xcell Electroporator (Hercules, CA, United States) with the following conditions: 625 kV, resistance at 600 Ω, capacitance of 25 μF. Cells were recovered overnight in YTF and plated the next day embedded in molten YTF 1.5% agar with 10 μg/mL thiamphenicol. Colonies appeared after 3 days. For generating the “3HB integration strain”, cells were grown in liquid YTF with thiamphenicol and 500 μg/mL 5-Fluoroorotic Acid (5FOA) based on previously reported protocols. Cells were then single colony plated in YTF agar with thiamphenicol and 5FOA, picked, and colony screened using PCR. To cure the thiamphenicol resistant plasmid, PCR confirmed colonies were then passaged in YTF without thiamphenicol until thiamphenicol sensitivity was restored.

### Analytical Techniques

Liquid fermentation products were processed via previously described methods. Briefly, samples were collected and filtered using Corning Costar Spin-X 0.45 μm (Corning, NY, United States) and routinely measured via HPLC, on a 1200 series Agilent (Santa Clara, CA, United States) Aminex HPX-87H column at 55° C. with a 4 mM H₂SO₄ mobile phase. Enzymatic determination of R-3-Hydroxybutyrate was performed on HPLC filtered samples using the “D-3-Hydroxybutyric Acid (β-Hydroxybutyrate) Assay Kit” from Megazyme (Ireland), using the manufacturer's instructions for the 96 well plate-based assay on a Tecan infinite M200 pro plate reader (Tecan Life Sciences, Switzerland). (R)-3-Hydroxybutyric acid and (S)-3-Hydroxybutyric acid were purchased from Sigma-Aldrich and used as standards from a 1 to 20 mM concentration. Optical density was measured using a Nanodrop (Thermofisher Scientific, Waltham, MA) at 600 nm. Carbon distribution was determined using only measured liquid components (i.e., Acetate, Ethanol, 3-Hydroxybutyrate, Fructose), measuring the mM and multiplying by the number of carbons in each product.

### Growth Conditions

Heterotrophic growth of the strains was carried out in 15 mL Falcon tubes (Fisher scientific) using a 4 mL YTF medium with/without the addition of 10 μg/mL thiamphenicol at 37° C. Cells from an overnight seed culture were added in a 1:5 ratio (1 mL culture into 4 mL YTF) and left for 3 days before sampling. Autotrophic growth with CO, CO₂, and H₂ was carried out using 250 mL Duran Pressure Plus bottles (DWK Life Sciences, USA) containing 50 mL YT medium (YTF without fructose). 10 mL of an overnight culture was added to the bottles. Bottles were sealed and aseptically flushed with a CO, CO₂, and H₂ mixture (70%/20%/10% CO/CO₂/H₂) for 3 min. The same gas mixture was then added to 6 PSI of pressure within the bottles. YT within the bottles was supplemented with 10 μg/mL thiamphenicol for growth with plasmid bearing strains. Bottle growth was carried out at 37° C. with 200 RPM shaking.

### Bioreactor Conditions

Autotrophic bioreactor growth was carried out using an Electrolab 2 L bioreactor, containing 1.70 L YT with 10 μg/mL thiamphenicol. 300 mL of an autotrophic seed culture was added, and growth was carried out with a CO, CO₂, and H₂ mixture (70%/20%/10% CO/CO₂/H₂) at a flow rate of 300 standard cubic centimeter per min (sccm) with a fine steel diffusion stone. The pH was maintained at 5.2 using 3 M NaOH and the temperature was kept at 37° C. using a heating wrap. Initial stirring was at 300 RPM which was increased to 500 RPM, followed by 900 RPM once cells began to grow based on OD₆₀₀. OD₆₀₀ and HPLC samples were taken daily.

### Enzyme Assays

Enzyme assays were performed based on previously reported procedures. Briefly, 50 mL of E. coli cells expressing C. kluyveri hbd1, C. ljungdahlii hbd2, and control vector pMTL83151 were harvested at mid-log phase and kept at −80° C. until the day of enzyme assays. The lysis was performed using a bead-beating method. The 3-Hydroxybutyryl-CoA dehydrogenase assay was performed under the following conditions: 100 mM potassium phosphate buffer (pH 6.5), 25 mM potassium citrate, 75 μM NAD(P)H, and 125 μM acetoacetyl-CoA in 200 μL 96-well plate with a BioTek Synergy Neo2 plate reader (BioTek Instruments, United States) at 6 second intervals for 10 min. Oxidation of NAD(P)H at 340 nm was used to follow enzyme activity, which is reported as μmol min⁻¹ mg⁻¹. To control for non-Hbd2 activity, we subtracted activity from E. coli cell free extract with pMTL83151, which was low (less than 0.01). Both hbd1 and hbd2 E. coli cell free extracts showed low activity without acetoacetyl-CoA (less than 0.01). Cell free extract protein was measured using a Bradford assay.

### Results

### 3HB Production Via an Acetoacetate Intermediate

3HB production via acetoacetate is a straightforward pathway, requiring three steps: thiolase (Thl) to condense two acetyl-CoA to acetoacetyl-CoA, acetate: acetoacetyl COA transferase (CtfAB) to transfer the CoA from acetoacetate-CoA to acetate, generating acetyl-CoA and acetoacetate, and 3Hbdh, which catalyzes the reduction of acetoacetate to 3HB (FIG. 1B). We were initially interested in the relatively understudied ctfAB/3hbdh pathway due to previous literature showing substantial unintended 3HB production during C. ljungdahlii acetone/isopropanol production via Thl and CtfAB. First, we wanted to determine whether C. ljungdahlii contains an effective and native enzyme to efficiently reduce exogenous acetoacetate to 3HB as previously suggested. A similar study had been done by spiking in acetone, whereby a native reductase efficiently converted +90% acetone to isopropanol, making it unnecessary to express a separate acetone reductase.

We therefore added 15 mM of acetoacetate to a growing culture of C. ljungdahlii to test endogenous reduction of acetoacetate. We measured about 5 mM 3HB, demonstrating that acetoacetate can be natively reduced to 3HB. However, as only about 5 mM of 3HB was detected, this suggests two thirds of the acetoacetate had been lost, and native acetoacetate conversion to 3HB is low.

Since it appears that acetoacetate reduction could be a limiting factor, we wanted to test various 3Hbdhs that could improve acetoacetate reduction. For the thiolase reaction, we chose the thiolase atoB from E. coli which showed good performance in C. acetobutylicum for butanol production. For the CoA transferase, we chose the ctfAB from C. acetobutylicum since this enzyme is well characterized and previously showed good functionality in C. ljungdahlii. We then tested putative 3HBDHs: one from C. difficile (CDIF630_02933, native sequence and a codon optimized version), one from Rhodobacter sphaeroides (Rsph17025_1507), and one from C. ljungdahlii (CLJU_c23220). We also note that the native C. ljungdahlii gene we overexpressed is not the gene responsible for 3HBDH activity. Nevertheless, the overexpression of CLJU_c23220 serves as a non-3hbdh overexpression control.

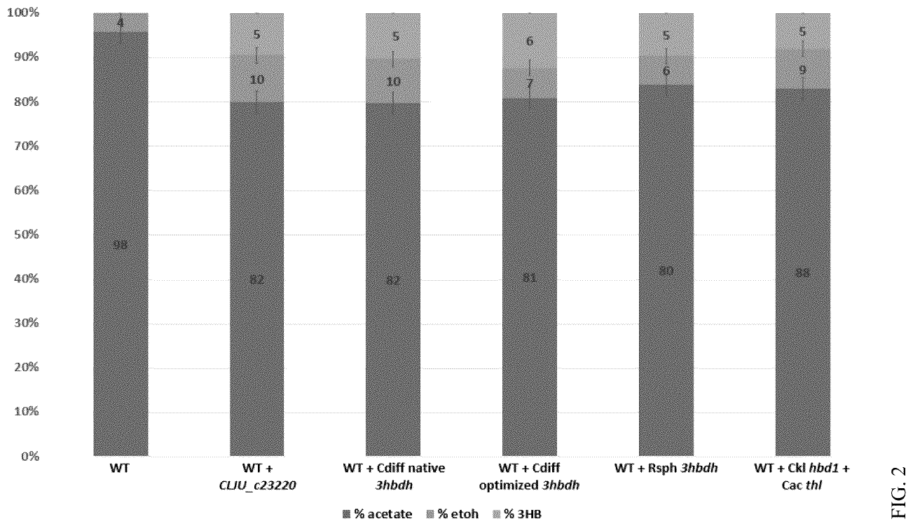

We also wanted to compare the hbd1 pathway to our ctfAB/3hbdh pathway due to a report of high 3HB titers in C. autoethanogenum. The hbd1 pathway uses the C. acetobutylicum thiolase (Cac thl) and hbd1 from Clostridium kluyveri (FIG. 1A). Expression of the pathways on each tested plasmid was driven by a C. ljungdahlii ferredoxin promoter (P_{fdx}) to allow for equal comparison. Plasmid bearing strains were grown in YTF medium containing fructose to observe heterotrophic production of 3HB. Final 3HB were determined after 3 days of growth (FIG. 2).

Results showed significant heterotrophic 3HB production via these both pathways. The transformed constructs demonstrated 5-6 mM of 3HB, which was comparable to the hbd1 based pathway (FIG. 2). This is consistent with that acetogenic heterotrophic 3HB production through this pathway is possible. Others have tried to express this pathway in both Clostridium coskatii and C. ljungdahlii, they were only able to demonstrate 3HB production in C. coskatii, as they did not detect 3HB production in C. ljungdahlii. Nevertheless, our data show that this pathway is in fact functional for 3HB production in C. ljungdahlii. Interestingly, explicit 3hbdh expression had only a marginal improvement on 3HB titers (2 mM difference between ‘Clj’ and ‘Cdiff’), despite our data showing native 3HBDH activity was poor.

### Creation of an Integrated 3HB Strain

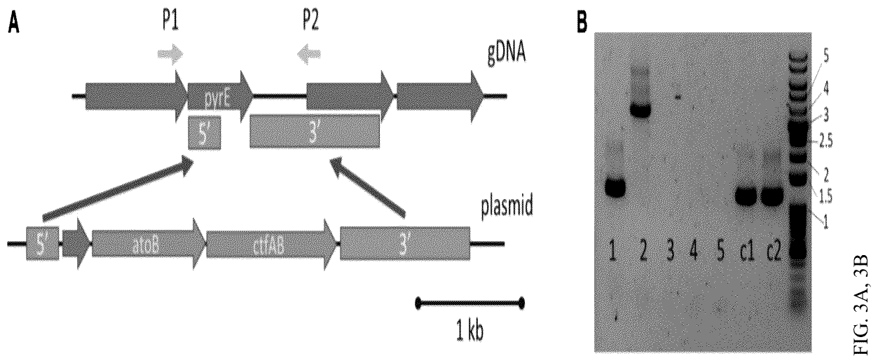

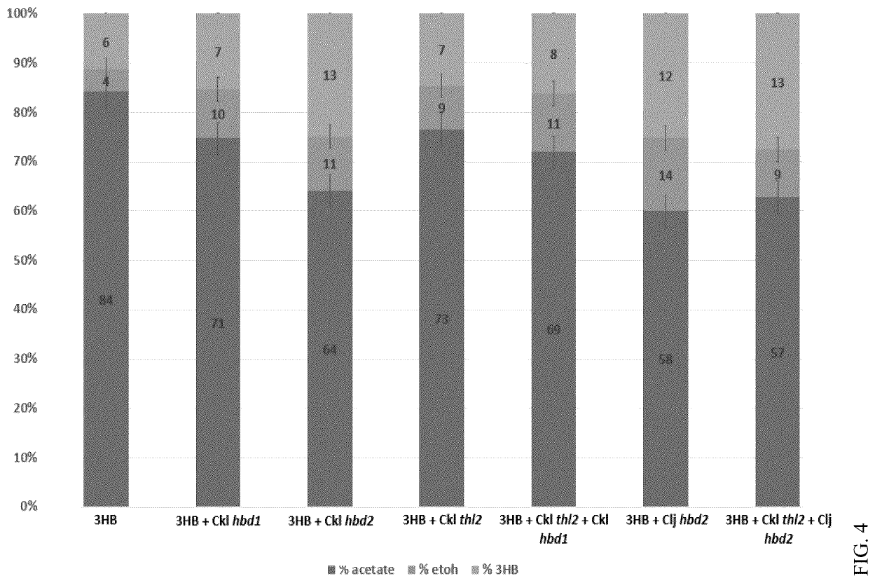

To improve 3HB titers, we coexpressed the ctfAB/3hbdh and hbd1 based pathways. We decided to integrate atoB and ctfAB into the pyrE locus to serve as base strain to allow us to test strategies for improving 3HB production with different expression constructs. Using 5-Fluoroorotic Acid (5FOA), homologous recombination can be used to place genes of interest into the pyrE locus, as pyrE+ cells are sensitive to 5FOA (FIG. 3). The genes atoB and ctfAB were successfully integrated into the genome, generating a strain that was 5FOA resistant. To cure the replicating plasmid, we subsequently passaged the strain on non-selective YTF media. We then isolated colonies that had become thiamphenicol sensitive, allowing for further transformations. PCR confirmation of integration was also carried out (FIG. 3B). This is referred to as the integrated 3HB strain and was confirmed to make 3HB (FIG. 4).

### Identification of a Native Hbd2 for Improving 3HB Production

Introduction of a CoA-transferase based acetone/isopropanol pathway noted significant 3HB production, even though acetoacetate should be rapidly decarboxylated to acetone in those strains. Our experiment indicated that native acetoacetate conversion to 3HB was not robust, with only a third of the acetoacetate converted to 3HB. In light of this evidence, it seems suspicious that previously reported 3HB was derived from acetoacetate, given poor 3HBDH activity and robust decarboxylation to acetone. Explicit 3hbdh expression did improve 3HB titers (FIG. 2), but not as much as we were expecting. While we were prospecting genes for overexpression, we noticed an annotated hbd2 in C. ljungdahlii and wondered whether it could be partially responsible for 3HB production. Transcriptomics evidence shows hbd2 (CLJU_c37300) is moderately expressed in C. ljungdahlii (˜300 FPKM), and we speculated this native gene could play a role in 3HB production. In contrast, hbd1 (CLJU_c23560) was not expressed (>1 FPKM) and therefore not pursued.

We generated a hbd2 expression construct similar to the ones expressed in C. autoethanogenum (FIG. 1A). Previous constructs by others only expressed thiolases and 3-hydroxybutyryl-CoA dehydrogenases but were very effective at generating 3HB in C. autoethanogenum. Our hbd2 construct used the ferredoxin promoter (P_{fdx}) to express a C. kluyveri thiolase (thl2) and the hbd2 from C. ljungdahlii. Since we were looking for further 3HB production enhancement, we first transformed the thl2 hbd2 construct into our integrated 3HB strain. Surprisingly, we found a 2.5-fold enhancement of 3HB production, from a 3HB carbon-yield of 11% (about 5 mM) to around 25% (about 12 mM) (FIG. 4).

We wondered whether C. kluyveri thl2 and C. ljungdahlii hbd2 could be efficient alone at making 3HB, so we transformed them into the wild-type background without the integrated thiolase and CoA transferase. Interestingly, we saw no 3HB production in this strain, suggesting that ctfAB is a critical gene, presumably to remove the CoA group from the 3HB-CoA generated by hbd2. To compare C. kluyveri hbd1, we also transformed hbd1 into a 3HB integrated strain, which showed improved 3HB production to 7.5 mM (about 2 mM over the 3HB parent) but was far inferior to the hbd2 strains (about 12 mM total). C. kluyveri also contains a hbd2 (CKL_2795), which we transformed as well, and performed similarly to the C. ljungdahlii hbd2 in 3HB titer (about 12 mM). This indicates that the hbd2 gene itself is important for increasing 3HB flux and is superior to the hbd1 gene in our system. In contrast, the plasmid expressing only thl2 in the integrated 3HB strain slightly improved 3HB to 7.5 mM, showing thiolase expression was not a major factor for 3HB production.

The C. kluyveri cofactor specificities of Hbd1 and Hbd2 are known from previous work (NADPH for Hbd1 and NADH for Hbd2), but unknown for C. ljungdahlii Hbd2. Using cell free extract of E. coli expressing C. ljungdahlii hbd2, we were able to measure a specific activity of 0.07 μmol min⁻¹ mg⁻¹, and we confirmed NADH specificity and acetoacetyl-CoA-dependence. C. kluyveri hbd1 E. coli extracts were used as an NADPH-specific control and measured a specific activity of 0.22 μmol min⁻¹ mg⁻¹. We attempted to determine Kₘ values for the C. ljungdahlii Hbd2 enzyme by varying either the NADH or the acetoacetyl-CoA concentrations. However, during this assay we noticed decreasing Hbd2 enzyme activity over several hours, inconsistent with the expected substrate loading conditions. We repeated the standard conditions of 75 μM NAD(P)H and 125 μM acetoacetyl-CoA with the hbd2 cell-free extracts, and we found markedly worse Hbd2 activity. In contrast, Hbd1 activity appeared to be intact, indicating that Hbd2 loses activity over time, even when kept on ice. We then lysed C. ljungdahlii cell-free extracts expressing hbd2 and found similar inconsistent activity results, suggesting that this was an Hbd2 related phenomena.

### Stereoisomer of Hbd2 3HB Production and Relative Contribution of Different Pathways

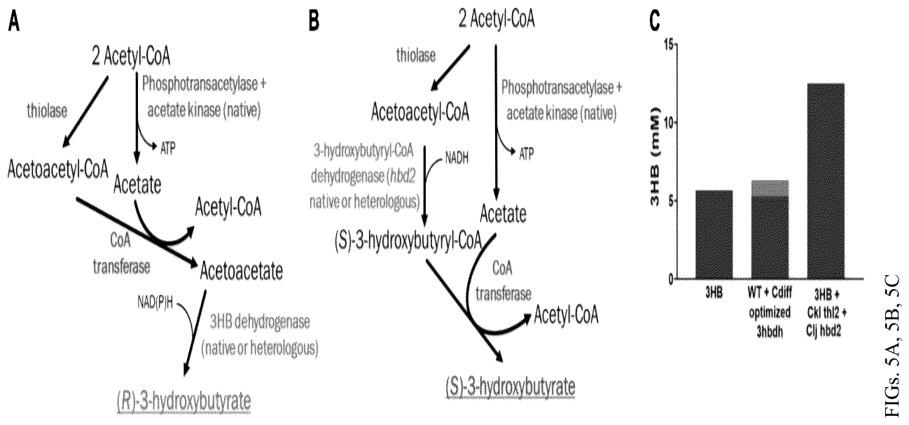

The stereoisomer of 3HB is important to determine, as the stereoisomer can determine its suitability for specific use cases and change bioplastics thermal/mechanical properties. Bioplastics physical properties can be driven and altered by the stereoisomer of the monomers. The relative contribution of the pathways in 3HB production can be determined by measuring the R or S form of 3HB. In the integrated 3HB strain, there are two possible pathways functioning to produce 3HB: the ctfAB/3hbdh and ctfAB/hbd2 pathway. The 3hbdh pathway produces the R form (FIG. 5A), while hbd2 produces the S form (FIG. 5B), and thus their relative contribution can be determined. While we quantified total 3HB via HPLC, we could not resolve the composition of 3HB stereoisomers, so we needed an alternative method.

To confirm that ctfAB/hbd2 was the main cause of 3HB production, versus the ctfAB/3hbdh endogenous pathway, we used a commercial R-3HB enzyme assay kit to detect R-3HB from the heterotrophic samples, see FIG. 2 and FIG. 4, for example. We tested three strains: the integrated 3HB strain, 3HB+Ckl thl2+Clj hbd2, and the wild type with ctfAB/3hbdh optimized from C. difficile. If the ctfAB/3hbdh pathway was the main pathway, we should get a detection from the enzymatic assay as it is specific for R-3HB, while S-3HB will not give a response. We did not detect any R-3HB in any of the integrated 3HB strains without 3hbdh overexpression less than 0.2 mM. In the wild type harboring the ctfAB/3hbdh with the optimized 3hbdh from Clostridium difficile, we detected around 1 mM R-3HB, which is around the magnitude of increase viewed in vivo with the added 3hbdh on the plasmid (FIG. 5C). This shows that the 3HB produced is mainly S-3HB, via the ctfAB/hbd2 pathway, and not R-3HB via acetoacetate reduction with 3HBDH. As a control, we added known amounts of R and S-3HB and confirmed only R-3HB is detected via this assay.

### Autotrophic Fermentation of Select Engineered Strains

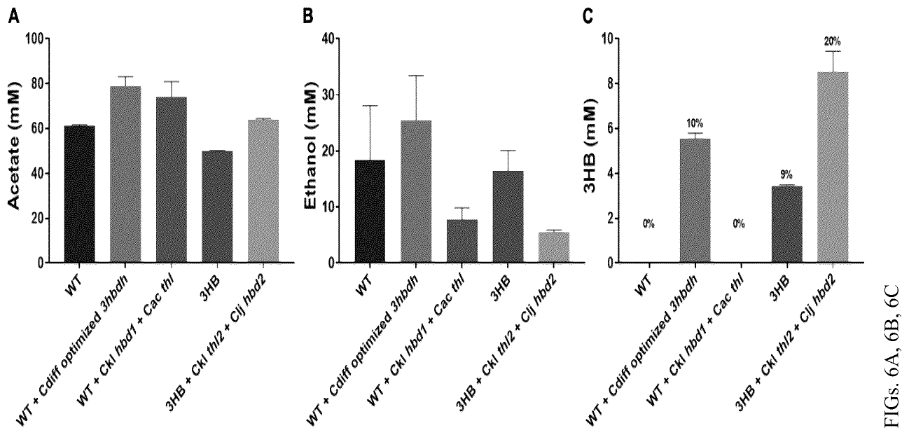

Next, we tested the performance of select strains growing on syngas. Previous experiments with acetogens showed drastically different 3HB production on syngas versus on sugar. For instance, in two previous works, high C. ljungdahlii 3HB titers (about 20 mM) were generated on fructose. However, that did not translate to autotrophic 3HB production (less than 1 mM). Thus, we took promising heterotrophic strains and tested their autotrophic performance in bottles (FIG. 6). Each strain was grown using a syngas mix of 70% CO, 20% CO₂, and 10% H₂ and product titers were analyzed after growth and gas consumption ceased. The hbd1 based pathway did not generate detectable 3HB. The integrated 3HB strain generated a small amount of 3HB, around 4 mM. The atoB, ctfAB, and 3hbdh strain generated the next most at about 6 mM, while the integrated 3HB strain+thl2+hbd2 strain generated the most at over 8 mM 3HB.

### Syngas Bioreactor 3HB Production

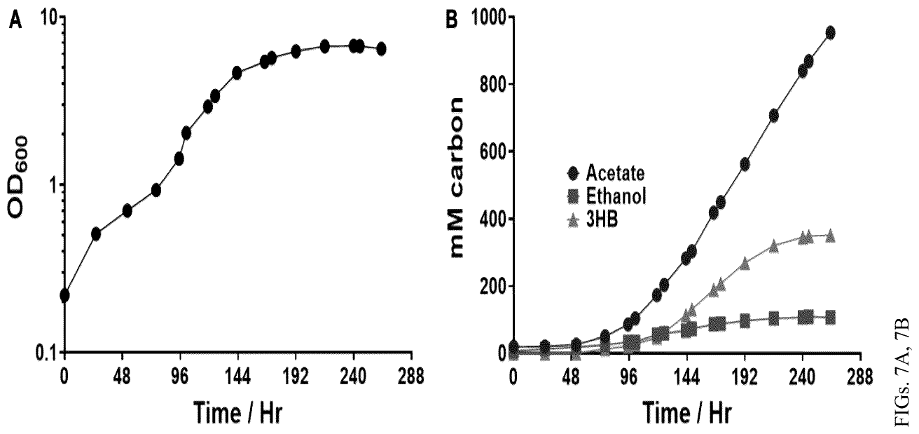

We then tested the autotrophic performance of the 3HB+thl2+hbd2 strain during bioreactor growth. We ran the strain under autotrophic conditions in a 2 L bioreactor, feeding 300 sccm of 70% CO, 10% H₂, 20% CO₂ maintained at pH 5.2 (FIG. 7). The gas fermentation was run for 263 h after which growth ceased. By the end, the strain had produced 476 mM acetate, 53 mM ethanol, and 88 mM 3HB, with a carbon distribution of 67%, 8%, and 25%, respectively. The highest 24-h 3HB productivity rate (that is, the max 3HB rate over a 24-h period) was 0.083 g/L/hr. This shows that the improvement in 3HB production also translates to autotrophic fermentations on syngas. It is also higher than the previously highest titer (28 mM) and yield (about 7%) reported in C. ljungdahlii from syngas fed-batch fermentation, which had focused on enhancing 3HBDH to improve 3HB titers.

### Discussion

As disclosed herein, our data shows that among the different 3HB pathways, ctfAB/hbd2 expression outperformed other pathways we tested in C. ljungdahlii. Furthermore, ctfAB/hbd2 was probably responsible for 3HB detection in previous reports targeting isopropanol/acetone production. Despite C. ljungdahlii native 3HBDH activity, acetoacetate conversion is poor, and seems unlikely to outcompete acetoacetate decarboxylation to acetone in those strains. Rather, our data indicates that the native hbd2 was likely responsible for the 3HB in those strains, and targeted overexpression of hbd2 can enhance the yield of 3HB over other tested pathways.

Without being bound by theory, Hbd2 (S)-3-hydroxybutyryl-CoA dehydrogenase likely explains the improved 3HB production. It has been previously shown in in vitro and in vivo systems that Hbd activity is key to driving high titers of both butanol and 3HB production, especially since thiolase condensation of acetyl-CoA is an endergonic reaction and downstream reactions are needed to pull the reaction forward. Interestingly, others have tested C. kluyveri hbd2 for 3HB production and it was found to underperform in a cell-free system compared to hbd1. The cell-free system used in these experiments did not have an explicit CoA-transferase, which may explain its in vitro underperformance. The hbd2 from C. beijerinckii was used in vitro to generate butanol, with superior performance versus hbd1. This was the only application examined for hbd2 and was not performed in vivo. The two in vitro studies provided conflicting evidence of the effectiveness of hbd2 vs. hbd1 and are the only biotechnical applications of hbd2 reported in the literature.

Without being bound by theory, another explanation could be the presence of CoA-transferase in the 3HB strain. We note that previous work in acetogens found good 3HB production on heterotrophic conditions but poor results on autotrophic conditions. It has been shown that acetyl-CoA levels can drastically change depending on heterotrophic versus autotrophic growth, so autotrophic flux towards 3HB may be improved by regenerating acetyl-CoA for (S)-3-hydroxybutyryl-CoA dehydrogenase activity. In E. coli, a CoA-transferase dependent 3HB pathway was elucidated by others where 3HB-CoA (generated by PhaB from Cupriavidus necator) would transfer the CoA to acetate, generating 3HB and acetyl-CoA. This pathway proved surprisingly efficient, generating a 3HB titer of 1 g/L. Relevant to acetogens, the 3HB titer was improved to 5.2 g/L with high acetate concentrations.

Little is known about hbd2 even though several Clostridia possess the gene. A BLAST search with C. ljungdahlii and C. kluyveri hbd2 indicates high identity to proteins in several Clostridia of scientific interest. Clostridium beijerinckii, Clostridium botulinum, Clostridium coskatii, Clostridium carboxidivorans and C. autoethanogenum generated high identity (greater than 70%) hits with hbd2. Interestingly, C. acetobutylicum only has an hbd1, and not hbd2. In contrast, the identities between hbd2 vs. hbd1 from C. kluyveri have about 40% amino acid identity to each other, suggesting that these enzymes are phylogenetically different. Butyryl-CoA synthesis genes often form an operon with hbd1, indicating a clear role in butyrate/butanol synthesis. In contrast, in both C. kluyveri and C. ljungdahlii, hbd2 are co-localized to genes unrelated to butyrate/butanol synthesis.

The C. kluyveri Hbd2 is NADH-linked, but its functionality is unknown. It is speculated to be important for redox balancing and chain elongation in C. kluyveri, where hbd1 is notably NADPH-linked. Almost nothing is known about hbd2 in C. ljungdahlii. We confirmed that C. ljungdahlii Hbd2 is NADH specific, like C. kluyveri Hbd2, but its function remains a mystery. C. ljungdahlii does not natively produce 3HB, PHB, butyrate, or butanol, and its genomic context doesn't appear to contain any obvious clues. It is moderately expressed in both heterotrophic and autotrophic conditions (FPKM 339 and 291, respectively) suggesting it could have an undetermined metabolic role.

C. ljunghdahlii has a number of functional genes that can natively catalyze 3HB production. It naturally converts acetoacetate to 3HB and has a highly functional Hbd2 that converts acetoacetyl-CoA to 3HB-CoA. 3HB has been produced in C. ljungdahlii and related acetogens, and although it was assumed that the heterologous expression of pathway components was responsible for 3HB production, native enzymes may also be playing a role in 3HB production. Published work expressing ctfAB in C. ljungdahlii assumed that 3HB was derived from acetoacetate reduction, but this 3HB could be from Hbd2 reducing acetoacetyl-CoA. One way to determine the relative contribution is through determination of the R vs. S stereoisomer via enzymatic assay analysis, which is a cheaper alternative than purchasing a chiral column. Furthermore, as different pathways have been expressed to produce 3HB in acetogens, 3HB titers could be increased by combining different pathway strategies into a single organism. As disclosed herein, in an embodiment, we show that both hbd2 and 3hbdh can be functional, as we did detect both S and R-3HB, but the 3hbdh contribution was low compared to hbd2.

Finally, this ctfAB/hbd2 pathway may have other advantages vs. previously described pathways. In our experimental conditions, hbd2 expression appears is superior to hbd1 and 3hbdh. Without being limited by theory, as a native acetogen/Clostridia derived enzyme, the Hbd2 may function better in its native host than heterologous enzymes. For instance, phaB has been tested in C. ljungdahlii with poor results, possibly due to compromised expression, despite good results in E. coli. Furthermore, the CoA transferase from 3-hydroxybutyryl-CoA to acetate regenerates acetyl-CoA, which may be important for acetyl-CoA concentrations and addressing ATP limitation when growing on H₂/CO₂/CO. For instance, previously described Hbd1-based 3HB production does not involve substrate-level phosphorylation, whereas the ctfAB/hbd2 described pathway would.

We have tested ctfAB from C. acetobutylicum and two hbd2, but BLAST screening shows a multitude of genes that could be tested. Greater 3HB yields could be gained by testing new genes and targeting acetyl-CoA related pathways, in particular acetate and ethanol. In vitro testing has recently proven successful in screening multiple 3HB genes. Furthermore, different 3HB pathways could potentially be combined in a single strain, as they do not appear to be incompatible and may improve 3HB yield/rate/titer. For instance, the hbd1 pathway in C. autoethanogenum generates significant amounts of acetate, which could be reassimilated when combined with ctfAB/hbd2. Additionally, while others have reported impressive titers of 3HB production based on the C. kluyveri Hbd1 in C. autoethanogenum, we were unable to repeat those results in C. ljungdahlii, suggesting there may be strain specific differences contributing to 3HB production. It is worth emphasizing that C. kluyveri Hbd2 is NADH-linked, while C. kluyveri Hbd1 is NADPH-linked. NAD(P)H redox differences may be a factor in 3HB production. Improved Clostridia product formation is often driven by changes to NAD(P)H metabolism. Beyond 3HB, Hbd2 could be important for other related products including PHB and longer chain fatty acids/alcohols (C4-C6).

Using methods and compositions of matter disclosed herein, hbd2 has been utilized in vivo for enhancing targeted product formation. Little is known about the native function of these genes, their biochemical characteristics/activity, and how they may be utilized to improve target product formation. Without being bound by theory, the native activity of Hbd2 could be unknowingly contributing to efforts to engineer 3HB/PHB/fatty acid/alcohol production in Clostridia, as hbd2 is commonly found in many Clostridia studied for metabolic engineering. As disclosed herein, these Hbd2 enzymes are useful for 3HB/PHB production. Additionally, Hbd2 catalyzes an important step in butanol/butyrate production, which are other value-added chemicals of interest that have been produced in acetogens. Furthermore, CoA-transferase based formation of 3HB appears to be a fruitful area of research for high 3HB production, especially considering that acetate formation is important for ATP synthesis in acetogens.

The foregoing discussion and examples have been presented for purposes of illustration and description. The foregoing is not intended to limit the aspects, embodiments, or configurations to the form or forms disclosed herein. In the foregoing Detailed Description for example, various features of the aspects, embodiments, or configurations are grouped together in one or more embodiments, configurations, or aspects for the purpose of streamlining the disclosure. The features of the aspects, embodiments, or configurations, may be combined in alternate aspects, embodiments, or configurations other than those discussed above. This method of disclosure is not to be interpreted as reflecting an intention that the aspects, embodiments, or configurations require more features than are expressly recited in each claim. Rather, as the following claims reflect, inventive aspects lie in less than all features of a single foregoing disclosed embodiment, configuration, or aspect. While certain aspects of conventional technology have been discussed to facilitate disclosure of some embodiments of the present invention, the Applicants in no way disclaim these technical aspects, and it is contemplated that the claimed invention may encompass one or more of the conventional technical aspects discussed herein. The following claims are hereby incorporated into this Detailed Description, with each claim standing on its own as a separate aspect, embodiment, or configuration.

We claim:

1. A non-naturally occurring Clostridium sp. comprising a heterologous ctfAB gene and a hbd2 gene.

2. The non-naturally occurring Clostridium sp. of claim 1 wherein the hbd2 gene expresses a (S)-3-hydroxybutyryl-CoA dehydrogenase having greater than 70% sequence identity to SEQ ID NO: 2.

3. The non-naturally occurring Clostridium sp. of claim 2 wherein the (S)-3-hydroxybutyryl-CoA dehydrogenase uses NADH as a co-factor.

4. The non-naturally occurring Clostridium sp. of claim 1 further comprising a heterologous atoB gene that is operably linked to the heterologous ctfAB gene.

5. The non-naturally occurring Clostridium sp. of claim 4 wherein the heterologous atoB gene that is operably linked to the heterologous ctfAB gene are both integrated into the chromosome of the Clostridium sp.

6. The non-naturally occurring Clostridium sp. of claim 3 wherein the specific activity of the (S)-3-hydroxybutyryl-CoA dehydrogenase is up to 0.07 mmol (S)-3-hydroxybutyryl-CoA per minute per milligram.

7. The non-naturally occurring Clostridium sp. of claim 1 wherein the Clostridium sp. is Clostridium ljungdahlii.

8. A method for making (S)-3-hydroxybutyrate comprising providing a carbon source to a solution comprising a non-naturally occurring Clostridium sp. comprising a heterologous ctfAB gene and a hbd2 gene.

9. The method of claim 8 wherein the hbd2 gene expresses a(S)-3-hydroxybutyryl-CoA dehydrogenase having greater than 70% sequence identity to SEQ ID NO: 2.

10. The method of claim 9 wherein the (S)-3-hydroxybutyryl-CoA dehydrogenase uses NADH as a co-factor.

11. The method of claim 8 wherein the non-naturally occurring Clostridium sp. further comprises a heterologous atoB gene that is operably linked to the heterologous ctfAB gene.

12. The method of claim 11 wherein the heterologous atoB gene that is operably linked to the heterologous ctfAB gene are both integrated into the chromosome of the Clostridium sp.

13. The method of claim 8 wherein the non-naturally occurring Clostridium sp. makes (S)-3-hydroxybutyrate at a rate up to is 0.083 g/L/hr for 24 hours.

14. The method of claim 8 wherein the non-naturally occurring Clostridium sp. makes (S)-3-hydroxybutyrate at up to a concentration of 88 mM.

15. The method of claim 8 wherein the non-naturally occurring Clostridium sp. makes (S)-3-hydroxybutyrate at a rate that is up to 2.5-fold greater than a naturally occurring Clostridium sp.

16. The method of claim 8 wherein the non-naturally occurring Clostridium sp. is Clostridium ljungdahlii.

17. The method of claim 9 wherein the specific activity of the (S)-3-hydroxybutyryl-CoA dehydrogenase is up to 0.07 mmol (S)-3-hydroxybutyryl-CoA per minute per milligram.

18. The method of claim 8 wherein the carbon source is syngas.

19. The method of claim 8 wherein the carbon source is sugar.

20. The method of claim 8 wherein the solution is anaerobic.


SEQUENCE LISTING (2 sequences)

SEQ ID NO: 1 - DNA, 942 bp, Clostridium ljungdahlii
  source 1..942: mol_type = genomic DNA
atgaatataaagaacatagcagttttaggaactggaactatgggacatggcatagcattactttctgcaaaagcaggattaaatgtagttatgtatggcagatctaatgcaagcttggaaagaggatttaataatataaaatctagcttaaatagtttaaaggctgcaggaaaattgtcagatgaaaaatgtgaagaaattttaagtaaaataaagggtgttaaaacaattcaagaagctgctaaagatgctgattttgtaatagaggcattagctgaaaacttagagctaaaacaggatatctttaggcagttggatgaaatttgttcaccagatgtagttttaactagtaatacttcaggacttagtccaacagctatagctaagaatacaaagtaccctgaaagagtggcaattgcccacttttggaatccaccacagctgattccattggtagaagtagtaccaggagaaaaaacatcacaggatacaatgaaaataatcatgaactgggttgaatttataggtaaaaaagctgttcgcatggaaaaggagtgtttgggttttatagggaataggcttcaacttgcacttcttagagaagctttgtatatagtagagcaaggatgggcaaaaccagaagaggtagacaaagctatggaatatggccatggaagaagattacctgttacaggcccattatgtagtgcagatttgggaggattggacatatttaacaatatatcttcatatttatttaaagatttgtgtaattatacgaaaccttctaagttaatgcaggataaagttgaagccggaaatttgggaagtaagagtggacaaggattttataattggacaccagaagttttagaaaagaaacaaaaagaacgtacagaagtacttttagactttttagagagggattctaggggaaaataa

SEQ ID NO: 2 - protein, 313 aa, Clostridium ljungdahlii
MNIKNIAVLGTGTMGHGIALLSAKAGLNVVMYGRSNASLERGFNNIKSSLNSLKAAGKLSDEKCEEILSKIKGVKTIQEAAKDADFVIEALAENLELKQDIFRQLDEICSPDVVLTSNTSGLSPTAIAKNTKYPERVAIAHFWNPPQLIPLVEVVPGEKTSQDTMKIIMNWVEFIGKKAVRMEKECLGFIGNRLQLALLREALYIVEQGWAKPEEVDKAMEYGHGRRLPVTGPLCSADLGGLDIFNNISSYLFKDLCNYTKPSKLMQDKVEAGNLGSKSGQGFYNWTPEVLEKKQKERTEVLLDFLERDSRGK